(19) United States
(12) Patent Application Publication
(10) Pub. No.: US 2022/0071478 A1
(43) Pub. Date: Mar. 10, 2022

(54) Anastomosis Testing Device and Method

(71) Applicant: THE REGENTS OF THE UNIVERSITY OF MICHIGAN, Ann Arbor, MI (US)

(72) Inventors: Curtis Bergquist, Ann Arbor, MI (US); Jeffrey Stephen Plott, Algonac, MI (US); Mark S. Cohen, Ann Arbor, MI (US)

(21) Appl. No.: 17/416,952

(22) PCT Filed: Dec. 20, 2019
(86) PCT No.: PCT/US19/67715

§ 371 Date: Jun. 21, 2021

Related U.S. Application Data
(60) Provisional application No. 62/782,752, filed on Dec. 20, 2018.

(51) Int. Cl. A61B 1/00; A61M 13/00; A61B 1/05; A61B 1/06; A61B 1/04

(52) U.S. Cl. CPC A61B 1/00082; A61M 13/003; A61B 1/05; A61B 17/1114; A61B 1/0676; A61B 1/043; A61B 1/00124

(57) ABSTRACT

A method and apparatus for testing the integrity of an anastomosis of a bodily lumen includes a multi-lumen tubular body having a first balloon that can be positioned proximally of an anastomosis, a second balloon that can be positioned distally of the anastomosis, and an aperture on the tubular body intermediate the first and second balloons. Once the first and second balloons are inflated, they define, in concert with the surrounding bodily lumen, a region that can be used to observe integrity of the anastomosis prior to conclusion of surgery. A fluid, such as air, is introduced through the aperture to the testing region, and a camera and lights provided on the instrument facilitate observation of any behavior of the fluid indicative of leaks in the anastomosis.

### CROSS-REFERENCE TO RELATED APPLICATIONS

This application claims the benefit of the filing date of, and priority to, U.S. Provisional Appl. No. 62/782,752, filed Dec. 20, 2018, the entirety of which is hereby incorporated by reference.

### BACKGROUND

### Field of the Invention

The disclosure relates to surgical procedures and, more particularly, to a method and apparatus for evaluating gastrointestinal tract anastomoses.

### Description of the Prior Art

The low anterior resection (LAR) is a surgical procedure performed to remove a portion of the colon located near the end of the colon and close to the rectum. Colon is removed for a variety of reasons including diverticulitis and cancer. The operation can be performed through a laparotomy incision (“open”), or laparoscopically (“minimally invasive”). In the LAR, the distal colon is removed, leaving the rectum distally and the remainder of the colon proximally. To give the patient an intact gastrointestinal tract from mouth to anus, a connection, or anastomosis, is made between the remaining colon and rectum. The anastomosis can be “hand sewn” using a variety of suture materials, or stapled, with a variety of different commercially available devices designed for creating new bowel anastomoses. Because an intervening segment of colon has been removed, the more proximal portion of the bowel which remains must be mobilized so that it will reach to the rectum. Natural connections between the bowel, other organs and the body wall are severed so that the bowel can reach the rectum in order to make the new anastomosis. It is important that the mobilized bowel reaches the rectum without disrupting its natural blood supply as this will damage the bowel and make the anastomosis more likely to leak. Anastomotic leak is undesirable because it leads to intra-intestinal contents, e.g. feces, spilling into the abdomen, causing potentially life-threatening infection. While the anastomosis techniques described above should create an anastomosis which is water-tight and without any holes through which intra-intestinal contents could leak, sometimes complications occur.

One risk or potential complication of gastrointestinal anastomosis procedures is that the stapler or other tools used to create the new connection may be defective and allow feces to leak out of the intestines and harm the patient. Another risk is that the blood supply of the intestines may be compromised; this complication may not be immediately apparent, leading to the anastomosis connection breaking down after the patient is out of the operating room. Yet another potential complication is bleeding inside the lumen of the intestine, which is not visible from the outside of the lumen (i.e., the surgeon's point of view) and may lead to life-threatening blood loss.

To minimize these risks, it is desirable to inspect a newly created bowel anastomosis. This can be done through a variety of existing techniques including rigid and flexible sigmoidoscopy. The anastomosis can also be “leak tested” by inflating the rectum with air, filling the abdomen with a liquid and occluding the proximal colon. The abdomen is then inspected for air bubbles which are presumed to be coming through a hole in an imperfect anastomosis. If bubbles are detected, the surgeon can re-make the anastomosis before leaving the operating room.

Unfortunately, it is at times challenging to assess a new anastomosis. Existing devices for checking newly-created anastomoses are expensive, time-consuming, require specialized skills to operate, do not allow for visual inspection and/or insufflate the entire bowel (which makes further surgery difficult). Additionally, some existing tools are reusable instruments, which can expose patients to infection, have high upfront costs and require maintenance. Currently available disposable instruments that allow for an intraluminal view lack screens for easy viewing, may not provide adequate air insufflation and cannot travel very far into the patient. Other devices which have screens require a large amount of insufflation, which is undesirable in the operating room; additionally, these devices are typically multi-use and require expensive maintenance and cleaning.

Desirably, a method and apparatus that overcome the foregoing problems would be available to conduct gastrointestinal anastomoses testing.

### SUMMARY

There is a need for an easy-to-use anastomosis testing device which allows for visual inspection of a new anastomosis and allows for pneumatic testing without inflating the entire bowel. The various aspects of the present disclosure address one or more of these needs and one or more of the problems noted above.

In various aspects, the present disclosure provides a new and improved method and apparatus specially adapted for testing and evaluating new anastomoses. In various aspects, the method and apparatus are especially suited for anastomoses of the gastrointestinal tract, including, for example, anastomoses of the bowel. It will be appreciated by those skilled in the art that even though various examples are directed to bowel anastomoses, the presently described method and apparatus may be suitable for use within any anastomoses of the gastrointestinal tract or other joining of lumens within the body.

### BRIEF DESCRIPTION OF THE DRAWINGS

FIG. 1A is a perspective view of one embodiment of the in-vivo portion of an anastomosis testing device of the present disclosure;

FIG. 1B is a distal end view of a device tip of the in-vivo portion of the anastomosis testing device of FIG. 1A;

FIG. 1C is a side cross section view of the device tip of FIG. 1B;

FIG. 2 provides a cross section, taken along lines 2-2 of FIG. 3, of the mid-portion of the tubing of one embodiment, between the control module portion (also referred to herein as a handle) and the in-vivo portion of the anastomosis testing device of FIG. 1A;

FIG. 3 provides a schematic view of the handle of one embodiment;

FIG. 4 provides a perspective view of one embodiment of the in-vivo portion of the device of the first embodiment, positioned within a patient to test a new anastomosis;

FIG. 5 is a plan view of a handle of a second embodiment of the present disclosure;

FIG. 6 is a perspective view of an alternate embodiment of the in-vivo portion of the anastomosis device of the present disclosure;

FIG. 7 is a plan view of a handle of a third embodiment of the present disclosure;

FIG. 8 is a perspective view of another alternate embodiment of the in-vivo portion of the anastomosis device of the present disclosure;

FIG. 9 is a plan view of a handle of a fourth embodiment of the present disclosure;

FIG. 10 is a perspective view of the in-vivo portion of the anastomosis device of FIG. 8; and

FIG. 11 is a plan view of a handle and external screen of the present disclosure, similar to that illustrated schematically in FIG. 3.

### DETAILED DESCRIPTION

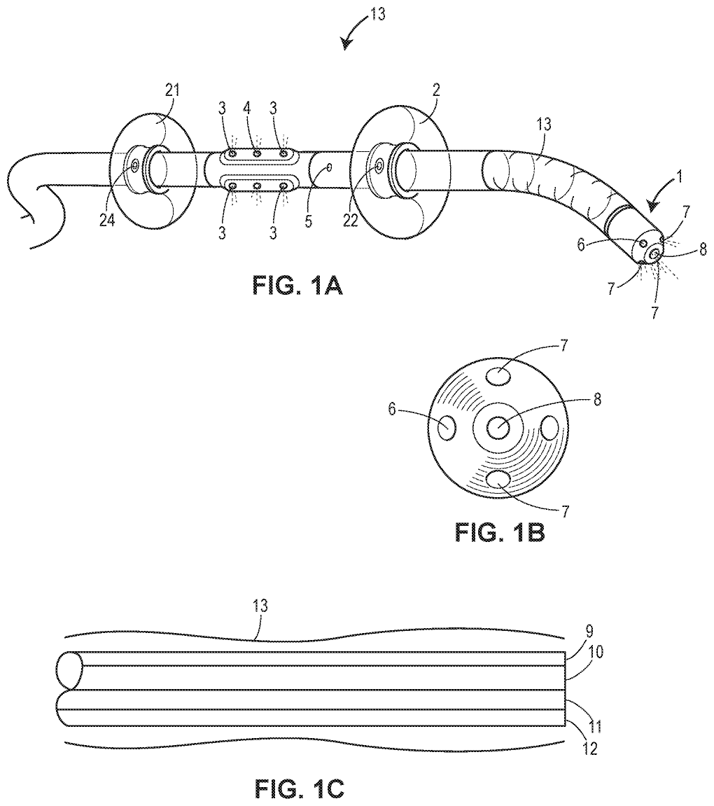

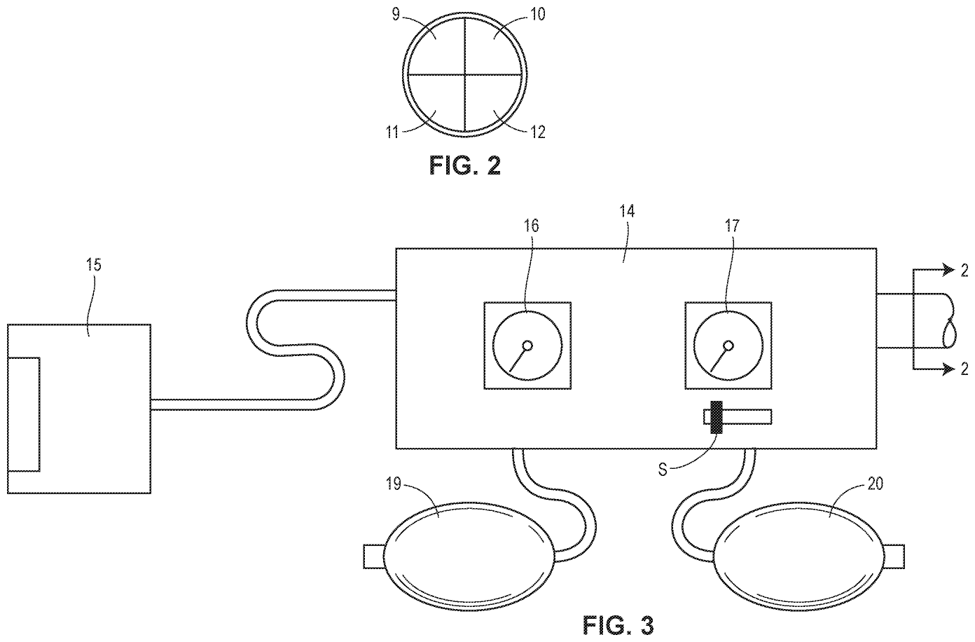

As shown in FIGS. 1 and 2, an apparatus according to one aspect of the disclosure includes a flexible, multi-lumen tubular body [13] of some desirable length. The distal tip of the apparatus [1] (i.e., the end that enters the patient and is furthest from the handle) may have an atraumatic shape to aid in movement through the bowels and be made of a soft polymeric material. To facilitate in-vivo navigation, the distal tip may be equipped with a camera [6], a light [7] (to allow navigation by transillumination of, for example, the bowel wall), and may further be provided with an aperture [8]. The aperture [8] provides an opening to a lumen [10] through which air or liquid may be expelled, such as for bowel insufflation. The lumen [10], or an additional lumen (not shown), may be used to aspirate fluids by being attachable, such as at a handle end, to a negative pressure source, such as standard operating room vacuum sources and tubing leading to collection canisters.

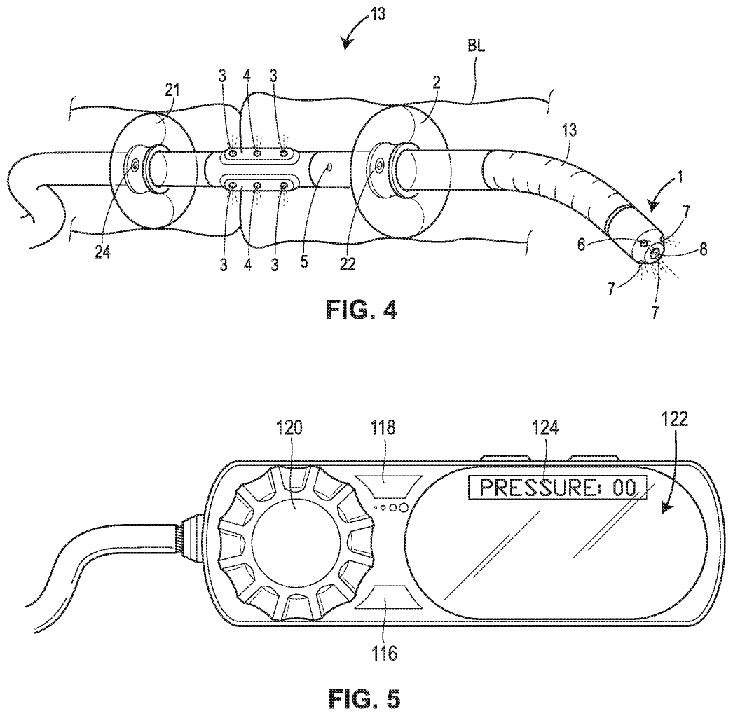

Proximal to the distal tip may be an anastomosis testing region, which includes: a first balloon [2]; a second balloon [21] axially spaced from the first balloon; at least one light source [3], at least one camera [4], and an opening [5] positioned between the first balloon [2] and the second balloon [21]. Each of the first balloon [2] and the second balloon [21] encircles the entirety of the tubing [13] and may be inflatable with air to create an air-tight seal in the bowel or other body lumen. As illustrated in FIG. 4, inflation of both the first and second balloons [2], [21] against the wall of the body lumen [BL] can create two air-tight seals, thereby forming an air-tight region within the body lumen [BL]. The light source(s) [3], camera(s) [4] and opening [5] (also referred to herein as an aperture) are located within this air-tight region. In this manner, when both balloons are inflated, the air-tight region between the balloons will be isolated from the rest of the gastrointestinal tract. As described in more detail below, when the balloons are inflated, air may be instilled into the isolated, air-tight section of the body lumen [BL] through the opening [5] and thereby enable pressure testing of the anastomosis.

In some embodiments, the first balloon [2] is located several centimeters back from (i.e., proximal to) the distal tip. In some embodiments, the opening [5] is located several centimeters back from the first balloon [2]. In some embodiments, the second balloon [21] is located several centimeters back from the opening [5]. In some embodiments, the handle is 10-100 cm back from the second balloon [21]. One or more cameras [4] and one or more accompanying light sources [3] may be positioned near the opening [5] and mounted around or embedded within) the circumference of the apparatus [1]. The interior of the tubing is comprised of or includes several lumens. A first lumen [9] contains the wiring necessary for the cameras and light sources. A second lumen [12] allows passage of air to the balloons [2], [21] to enable balloon inflation. The tubing [13] includes inflation ports [22], [24], in fluid communication with the second lumen [12] and with an interior of the respective balloons [2], [21]. While inflation ports [22], [24] may share a common lumen [12], an alternative is that each inflation port [22], [24] is serviced by a separate respective lumen. A third lumen [11], also referred to herein as an insufflation lumen, terminates at the opening [5] between the two balloons is configured for allowing the passage of air or liquid through its lumen and the opening [5] into the isolated section of the body lumen. A fourth lumen [10] extends the length of the apparatus [1], terminating at the aperture [8] at the tip of the apparatus and enabling the passage of air or liquid through the apparatus [1]. Additional lumens (not shown) could also be included to incorporate pull wires which can act to articulate the distal tip [1] of the device to aid in in-vivo navigation.

A proximally located control module, or handle [14], is provided in FIG. 3. The handle [14] is not configured or intended to enter the patient. The handle [14] may include a first connector (herein referred to as a balloon connection) configured to connect the second lumen [12] to a pump [19] to inflate the balloons. The second lumen [12] is in fluid communication with a first balloon inflation port in fluid communication with an interior of the first balloon [2], and is in further fluid communication with a second balloon inflation port in fluid communication with an interior of the second balloon [21]. The first and second balloons [2], [21] are preferably inflated and deflated simultaneously through a common lumen [12], but as indicated above, independent inflation lumens could be provided, respectively, to each inflation port [22], [24] to permit the first and second balloons [2], [21] to be inflated and/or deflated independently of one another. The second lumen [12] may also be coupled to or include a gauge [16] to measure the pressure inside the balloons and a valve in order to expel air from the balloons and release the pressure. The handle [14] may also include a second connector (herein referred to as an aperture connection) configured to connect to a fluid source (e.g., intravenous tubing or a pump) in order to instill air or liquid into the third lumen [11] or fourth lumen [10]. In some embodiments, the aperture connection is a Luer lock or other connection configured to connect to standard intravenous fluid tubing. In some embodiments, the aperture connection connects to a second pump [20], which is independent of pump [19], and is coupled to or includes a gauge [17] to measure the pressure and a valve to release accumulated pressure. In some embodiments, the handle [14] includes a switch S that allows a user to choose between directing fluid received at the aperture connection to either the opening [5] or the aperture [8]. Alternatively, a single pump or other fluid source can be used to selectively introduce and expel air (or liquid) to not only the balloons [2], [21], but also to the aperture [8] and/or the opening [5].

The wiring in the first lumen [9] connects to a cord and plug, which are configured to form an electrical connection to a signal processing device [15]. Through this signal processing device, camera images are displayable on one or more screens. The one or more screens may show the images obtained from each of a plurality of cameras. A switch may be provided to power the lights. Another switch may be provided to change the camera and lighting for the fluorescein angiography function.

The apparatus is used for pneumatically testing new anastomoses during gastrointestinal surgery following removal of an undesirable portion of the gastrointestinal tract and reconnection of the remainder of the tract. The operations where this procedure occurs may be ‘open’ through a large incision in the abdominal wall, or laparoscopic, which utilizes smaller incisions, long instruments designed to work through ports and a camera for viewing.

A bowel anastomosis is shown in FIG. 4. A portion of diseased bowel has been removed leaving two blind pieces of bowel remaining which are closed by a line of surgical staples or a surgical clamp. The anastomosis is fashioned either sewing ‘by hand,’ or by a suitable tool, such as specifically designed surgical staplers. For a connection between the colon and rectum, the anastomosis may be ‘end to end,’ such that the cut end of the colon is joined with the cut end of the rectum. Specific surgical staplers can create such ‘end to end’ anastomoses of a fixed size. Other anastomoses may be created from the small bowel to the large bowel, again either ‘hand sewn’ with a series of permanent sutures or using a surgical stapler device. These anastomoses between bowel of unequal diameter may connect ‘side to side’ or ‘end to side,’ depending upon the orientation of the bowels or the surgeon's preference. For any anastomosis, it is essential that anything passing through the interior lumen, such as feces, cannot leak out of the lumen through a defect. It is also essential that the blood supply to the gastrointestinal tract is not compromised by excessive tension or twisting.

As shown in FIG. 4, the apparatus may be inserted into the lumen of the gastrointestinal tract and advanced to the location of the anastomosis. The apparatus provides air leak detection, visualization and blood-flow assessment for both open and laparoscopically created anastomoses. The apparatus features two balloons [2], [21] spaced back from the tip which occlude the gastrointestinal lumen and permit pneumatic leak testing. While the balloons [2], [21] are illustrated as two separate balloons, each of which may be toroidal or of some other shape that is sufficient to seal a surrounding bodily lumen upon inflation, it is recognized that the balloons [2], [21] could alternatively take the form, collectively, of two bulbous ends of a single differentially-expandable balloon, such as a peanut-shaped balloon, that permits sealing on both sides of the anastomosis, and has a dwell portion intermediate the bulbous ends, the dwell portion including an opening [5] to permit insufflation of air to an area between the bulbous ends that includes the anastomosis. The distal tip [1] of the device is soft to avoid damage to the bowel wall. The overall apparatus is long and flexible so that it may be passed several centimeters into the patient. The distal tip [1] has a camera [6], at least one light source [7], and lumen for the passage of air or other desired substance through the aperture [8]. Between the two balloons there are additional cameras [4] and light sources [3] for inspecting the anastomosis, and if desired, performing fluorescein angiography. There is also an opening [5] between the two balloons to allow the insufflation of air. The wiring [9] and air channels are contained within the single flexible tubular body [13] and interact with the user through different portions of the handle [14]. An electrical cord provides power to the lighting and allows for viewing the cameras [15].

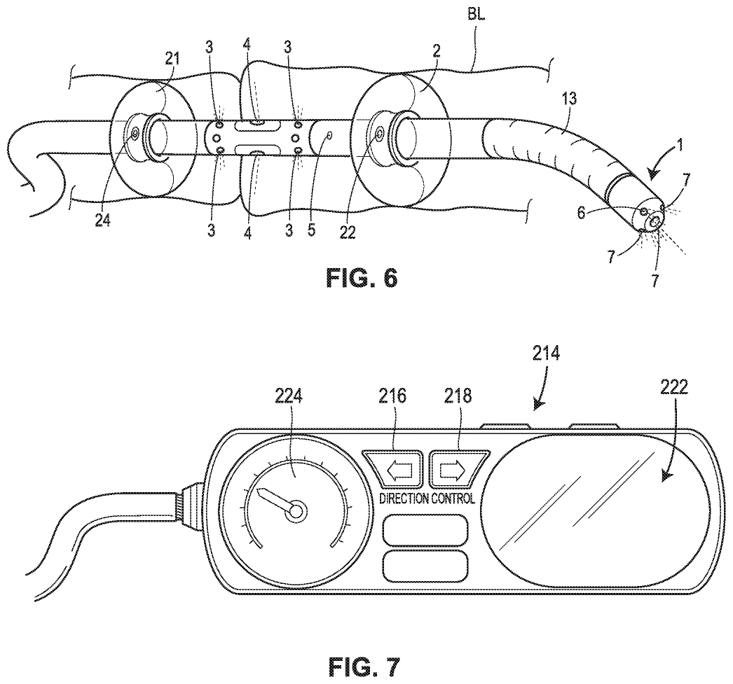

An alternative arrangement of light sources [3] and cameras [4] for the in-vivo portion of the anastomosis testing device is illustrated in FIG. 6. According to this embodiment, the light sources [3] include a first ring of lights and a second ring of lights [3]. Three cameras [4] are provided, preferably at 120° intervals, around the tube [13], which can be used to facilitate viewing of the entirety of the intralumenal circumference of the anastomosis.

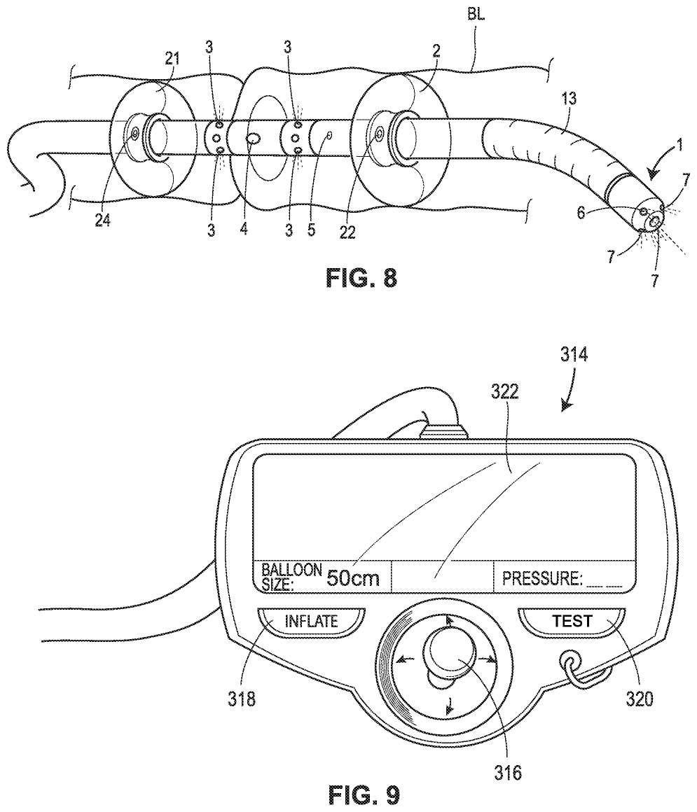

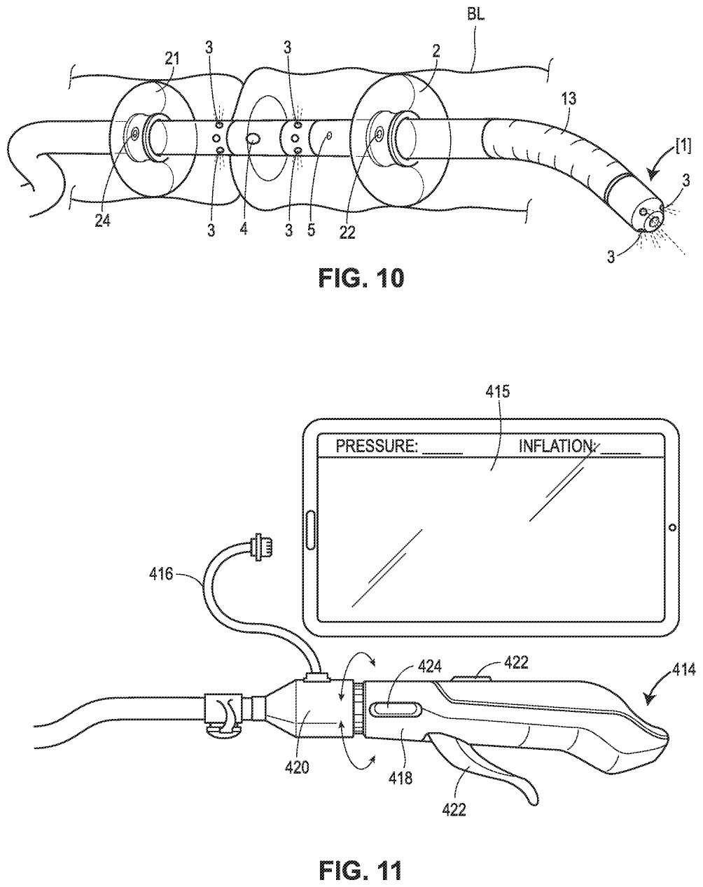

A further alternative arrangement of light sources [3] and cameras [4] for the in-vivo portion of the anastomosis testing device is illustrated in FIGS. 8 and 10. In this embodiment, the camera [4] between the balloons [2], [21] is a single, rotatable camera [4]. As in the embodiment of FIG. 6, the light sources [3] may include a first ring of lights and a second ring of lights. The camera [4] of this embodiment can preferably rotate through a full 360°, facilitating viewing of the entirety of the intralumenal circumference of the anastomosis.

In some embodiments, at least the in-vivo portion of the device arrives in sterile packaging and is intended for disposal after use in a single patient.

Turning to FIG. 5, an alternate embodiment of a user control module, or handle [114], is illustrated. According to this embodiment, the handle [114] includes control buttons [116], [118], a control wheel or knob [120], and a screen [122] that can display video obtained from the cameras [4], [6]. Data such as pressure within a pressurized test region (i.e. between inflated balloons [2], [21] and across an anastomosis) may appear on a digital pressure gauge [124] of the screen [122]. The data displayed by the digital pressure gauge [124] may be measured air pressure, for example in units of psi or KPa, or may be data in a more intuitive, binary format, such as green for inflation pressure or test pressure being in an acceptable range (indicative of, for example, the balloons [2], [21] being in a fully inflated condition, or the anastomosis being free of leaks), and red for inflation pressure not being acceptable (indicative of, for example, the balloons [2], [21] being only partially inflated, or a potential leak in the anastomosis).

The control buttons [116], [118] and the control wheel or knob [120] remotely control articulation of the distal tip [1]. The control wheel [120] may, through a network of cables (not shown) passing through the multi-lumen tube [13], have a direct mechanical linkage with a steering mechanism at the distal tip [10] to provide deflection, and therefore navigation. Alternatively, the control wheel and control buttons [116], [118] may control of articulation of the distal tip [10] through electromechanical impulses and signals. However, mechanical linkages provide the benefit of direct tactile feedback.

Turning to FIG. 7, another embodiment of a user control module, or handle [214], is illustrated. This embodiment includes control buttons [216], [218], a screen [222], and a pressure gauge [224]. The control buttons [216], [218] may provide directional control to at least the distal tip [1]. According to this embodiment, the pressure gauge [224] may be analog as opposed to digital (or may be a digital display of an analog-style pressure gauge), and may display pressure in both KPa and psi units.

Another embodiment of a user control module, or handle [314], is illustrated in FIG. 9. According to this embodiment, a joystick [316], similar to that of an electronic gaming system controller, is provided to control articulation of at least the distal tip [1]. The handle [314] may be provided with dedicated buttons [318], [320]. One of the buttons [318] may be designated “INFLATE”, and which, as the name implies, upon depression, results in introduction of air to the balloons [2], [21] to inflate them with determined volume (or pressure) of air. The other button [320] may be designated “TEST”, and which, upon depression, results in introduction of air or liquid through the opening [5] to test the integrity of the anastomosis. The handle [314] also includes a screen [322] to enable viewing of images obtained by the camera(s) [4] of an associated in-vivo portion of the anastomosis testing device, and/or the screen [322] may display fluorescein angiography signals. The screen [322] may serve as a graphical user interface and be provided with one or more permanently designated areas [322a], [322b] thereon to display desired user-manipulable data, such as balloon size and pressure. By selecting a desired balloon size and/or pressure, the amount of air introduced upon actuation of the designated “INFLATE” button [318], and air or other fluid introduced when pressing the designated “TEST” button [321], can be set.

A further embodiment of a user control module, or handle [414], is illustrated in FIG. 11. As was the case with the handle [114] discussed above and illustrated schematically with respect to FIG. 3, this handle [414] does not have an integral screen, but rather, a data cable [416] delivers information to an external screen [415]. The handle may have a main body portion [418] axially aligned with a hub portion [420], the main body portion [418] preferably being rotatable relative to the hub portion [420], with rotation of the main body portion [418] relative to the upper portion resulting in corresponding articulation of the distal tip [1]. The handle [414] may be further provided with a trigger [422] to initiate and cease inflation of the balloons [2], [21], and/or to initiate and cease introduction of air or other fluid through the opening [5] to test the integrity of the anastomosis once the balloons [2], [21] are inflated. Designated regions of the external screen [415] may display pressure and inflation of the balloons [2], [21], and/or of the region of the anastomosis between the two inflated balloons [2], [21]. Additionally, the external screen [415] receives and displays at least one of video or fluorescein angiography signals from the camera(s) [4], [6]. One or more buttons [422], [424] can be provided to, for example, lock the trigger [422] so as to stop introduction of further air into either the balloons [2], [21] or to stop further introduction of air or liquid into the region of the anastomosis, or to toggle a valve (not shown) to changeover from balloon inflation/deflation to introduction or evacuation of air or liquid through the opening [5]. At least one of the buttons [422], [424], or an additional button, switch, or virtual interface, may be provided to control at least one of the camera(s) [4], [6] and the one or more lights [3].

Additionally, it should be noted that the balloons [2], [21] need not necessarily be integrally combined with the tubing [13] and articulating distal tip [1]. Rather, the balloons [2], [21], together with the camera [4], and a lumen having an aperture [5], and optionally, one or more lights [3], could be slid over, and retained on, an existing endoscopic device or bronchoscope, and used to perform anastomosis testing in accordance with the present disclosure. Such a configuration could advantageously ease navigation to the target site, by not having to navigate an instrument with the balloons connected. Rather, in one potential method of testing preparation, the endoscopic device or bronchoscope could be navigated to the desired location, past the anastomosis, then the balloons [2], [21], together with the camera [4] and lumen having an aperture [5], with optional light(s) [3], could be slid along, and secured to the endoscopic device or bronchoscope in-vivo, once at the desired position within the anastomosis. An additional advantage of such an embodiment would be that the balloons [2], [21], camera [4], lumen with an aperture [5], and optional light(s) [3], or some subset thereof, could be sterilized and packaged for single-patient use.

Method of Operation

After the creation of a new gastrointestinal anastomosis, the device is inserted into whichever natural orifice is nearest, whether it be ostomy, mouth or rectum. The device is advanced to the anastomosis under direct vision and possibly guidance of the surgeon and while watching the camera output on-screen. Once the device is near the anastomosis, the balloon [2] may be inflated [19] in order to create a seal within the lumen of the bowel. Visual inspection of the bowel distention as well as direct pressure measurements [16] [17] will guide the level of balloon inflation. The other portion of the bowel can be occluded with the surgeon's hand or surgical instrument, such as a suitable clamp. Air is then insufflated through the end of the device via introduction of air through the aperture [8]. The camera on the tip [6] allows the surgeon to inspect for bleeding and switching modes allows for fluorescein angiography. Alternatively, the device may be passed across an anastomosis such that the anastomosis lays between the two balloons [2], [21], as in FIG. 4. The balloons are then inflated as in the other example and air is insufflated between the balloons, thus pneumatically testing the anastomosis. The cameras between the balloons [2], [21] allow for inspection of the anastomosis and possibly fluorosceine angiography. After testing is complete, air is evacuated and the balloons are deflated before moving the device out of the patient.

The steps for testing are:
If used without crossing an anastomosis:
   Passing device to the anastomosis using camera guidance;
   Clamping the bodily lumen at a location spaced from the anastomosis;
   Injecting the patient with fluorescent dye;
   Switching the light/camera to angiography to view blood flow;
   Returning to normal camera optics;
   Inflating one or more balloons;
   Insufflating air or a fluid via the lumen at the distal tip of the device;
   Look for air or fluid leak from the anastomosis from the surgical field;
   Deflate the balloon(s);
   remove device.

It should be recognized that, if used in conjunction with a clamp or other suitable means of occluding the bodily lumen at a location away from the anastomosis, only a single balloon needs to be provided on the device, or if two balloons are provided, only one of the balloons needs to be inflated. If desired, a device with a single opening [5] between two balloons, but no aperture [8] at the distal tip [1] of the device, could still be used in such a manner, by only inflating the balloon [21] on an opposite side of the anastomosis from the side that is clamped or otherwise occluded, with both the opening [5] and the anastomosis intermediate the clamped or otherwise occluded section and the inflated balloon [21].

If crossing the anastomosis:
Passing device past the anastomosis using camera guidance such that the two balloons rest on either side of the anastomosis
Injecting the patient with fluorescent dye
Switching the light/camera to angiography to view blood flow
Returning to normal camera optics
Inflating the balloons
Insufflating air or a fluid via the opening between the balloons
Look for air or fluid leak from the anastomosis from the surgical field
Deflate the balloons, remove device

Although the invention has been described in a preferred embodiment with a certain degree of particularity, it will be understood that the present disclosure has been made only by way of example and that various changes may be resorted to without departing from the true spirit and scope of the invention as hereinafter claimed. It should be understood that while various handle configurations and various in-vivo portions of an anastomosis testing device are disclosed herein, they may be used interchangeably with one another, and variations may be made thereto, that are still considered within the scope of the appended claims.

What is claimed is:

1. A surgical apparatus for testing integrity of a gastrointestinal bowel anastomosis, comprising:
a flexible, multi-lumen tubular body having a distal tip;
a first balloon encircling the multi-lumen tubular body and in fluid communication with an inflation lumen of the multi-lumen tubular body;
the flexible, multi-lumen tubular body further including an aperture, the aperture in fluid communication with an insufflation lumen of the multi-lumen tubular body; and
a handle in communication with each of the lumens of the multi-lumen tubular body, the handle including
a first port in fluid communication with a fluid source for supplying air to the inflation lumen; and
a second port in fluid communication with the fluid source, or an additional fluid source, for supplying at least one of a liquid or a gas to the insufflation lumen.

2. The surgical apparatus of claim 1, further including a second balloon encircling the multi-lumen tubular body, the second balloon spaced axially from the first balloon along the multi-lumen tubular body and being in fluid communication with the inflation lumen of the multi-lumen tubular body; and wherein the aperture is intermediate the first balloon and the second balloon.

3. The surgical apparatus of claim 1, the handle further including:
a balloon pressure gauge for monitoring pressure of air supplied by one of the at least one fluid sources.

4. The surgical apparatus of claim 1, the handle further including:
an insufflation pressure gauge for monitoring pressure of at least one of liquid or gas supplied by the one of the at least one fluid sources.

5. The surgical apparatus of claim 2, further comprising a camera disposed on an exterior of the multi-lumen tubular body intermediate the first balloon and the second balloon, the multi-lumen tubular body including wiring to at least one of provide power to and deliver video signals from the camera.

6. The surgical apparatus of claim 5, further comprising one or more lights disposed on an exterior of the multi-lumen tubular body intermediate the first balloon and the second balloon, the multi-lumen tubular body including wiring to selectively power the one or more lights.

7. The surgical apparatus of claim 5, wherein the handle further comprises a switch to control at least one of the camera and the one or more lights.

8. The surgical apparatus of claim 5, wherein the handle is in electronic communication with an output screen to which at least one of video or fluorescein angiography signals are received from the camera and displayed.

9. The surgical apparatus of claim 5, wherein the camera is rotatable about a circumference of the multi-lumen member.

10. The surgical apparatus of claim 5, wherein the camera is a first of a plurality of cameras disposed at regular intervals from one another about a circumference of the multi-lumen member.

11. The surgical apparatus of claim 10, wherein each of the cameras of the plurality of cameras is disposed at 120° from a next adjacent of the plurality of cameras.

12. The surgical apparatus of claim 1, wherein the handle includes at least one shutoff valve that, when closed, maintains the at least one balloon in its then-current state of inflation.

13. The surgical apparatus of claim 1, wherein each of the first port and the second port is a luer lock.

14. The surgical apparatus of claim 1, wherein the handle further includes a control mechanism to actuate the distal tip of the flexible, multi-lumen tubular body.

15. The surgical apparatus of claim 1, wherein the distal tip includes a camera and at least one light.

16. A method of testing integrity of an anastomosis within a bodily lumen, comprising:
introducing a flexible, multi-lumen tubular body into a bodily lumen of a of a patient in which an anastomosis has been created, the flexible, multi-lumen tubular body including a first balloon encircling the multi-lumen tubular body and in fluid communication with an inflation lumen of the multi-lumen tubular body; a second balloon encircling the multi-lumen tubular body, the second balloon spaced axially from the first balloon along the multi-lumen tubular body and being in fluid communication with the inflation lumen of the multi-lumen tubular body; the flexible, multi-lumen tubular body further including an aperture intermediate the first balloon and the second balloon, the aperture in fluid communication with an insufflation lumen of the multi-lumen tubular body;
positioning the flexible, multi-lumen tubular body within the colon such that one of the first balloon and the second balloon is positioned proximally of the anastomosis and the other of the first and second balloon is positioned distally of the anastomosis;
inflating the first balloon and the second balloon, thereby creating a seal within the bodily lumen of the patient; and
introducing a fluid in the form of at least one of a liquid or a gas through the insufflation lumen.

17. The method of testing integrity of an anastomosis of claim 16, wherein the flexible, multi-lumen body is in communication with a handle, the handle including a first port in fluid communication with a balloon inflation pump for supplying air to the inflation lumen; and a second port in fluid communication with an insufflation pump for supplying at least one of a liquid or a gas to the insufflation lumen, and in inflating the first balloon and the second balloon, operating the balloon inflation pump to supply air to the inflation lumen.

18. The method of testing integrity of an anastomosis of claim 17, and in introducing a fluid in the form of at least one of a liquid or a gas through the insufflation lumen, operating the insufflation pump to supply at least one of a liquid or a gas to the insufflation lumen.

19. The method of testing integrity of an anastomosis of claim 16, wherein the flexible, multi-lumen body has a camera disposed on an exterior thereof intermediate the first balloon and the second balloon, further comprising viewing at least one of video and angiography signals from the camera.

20. The method of testing integrity of an anastomosis of claim 16, wherein the flexible, multi-lumen body has a camera disposed on a distal tip end thereof, further comprising viewing at least one of video and angiography signals from the camera.

21. The method of testing integrity of an anastomosis of claim 17, further comprising manipulating a control mechanism of the handle, the control mechanism being in at least one of mechanical and electronic communication with a distal tip end of the flexible, multi-lumen body so as to control articulation of the flexible, multi-lumen body.

22. The method of testing integrity of an anastomosis of claim 21, wherein manipulating the control mechanism of the handle includes rotating a main body portion of the handle relative to a hub.

23. The method of testing integrity of an anastomosis of claim 16, further comprising sliding the flexible, multi-lumen tubular body over, and securing it to, an endoscopic device.

24. The method of claim 23, wherein in sliding the flexible, multi-lumen tubular body over, and securing it to, the endoscopic device, the endoscopic device is already located within the bodily lumen, in proximity to the anastomosis to be tested.

25. A method of testing integrity of an anastomosis within a bodily lumen, comprising:
introducing a flexible, multi-lumen tubular body into a bodily lumen of a patient in which a gastrointestinal anastomosis has been created, the flexible, multi-lumen tubular body including at least one balloon encircling the multi-lumen tubular body and in fluid communication with an inflation lumen of the multi-lumen tubular body; the flexible, multi-lumen tubular body further including an aperture, the aperture in fluid communication with an insufflation lumen of the multi-lumen tubular body;
positioning the flexible, multi-lumen tubular body within the colon such that the at least one balloon is positioned proximally of the anastomosis;
clamping the bodily lumen at a location opposite the anastomosis from the at least one balloon;
inflating the at least one balloon, thereby creating a seal within the bodily lumen of the patient; and
introducing a fluid in the form of at least one of a liquid or a gas through the insufflation lumen.
